(19) United States
(12) Patent Application Publication
(10) Pub. No.: US 2021/0068648 A1
(43) Pub. Date: Mar. 11, 2021

(54) Biofilm Reveal Device

(71) Applicant: The Procter & Gamble Company, Cincinnati, OH (US)

(72) Inventors: John Giuseppe Schilleci, Sao Paulo (BR); Heitor Jose Leite Silva, Sao Paulo (BR)

(21) Appl. No.: 17/016,428

(22) Filed: Sep. 10, 2020

Related U.S. Application Data
(60) Provisional application No. 62/898,655, filed on Sep. 11, 2019.

(51) Int. Cl. A61B 1/247; A61B 1/04; A61B 1/06

(52) U.S. Cl. CPC A61B 1/247; A61B 1/06; A61B 1/04

(57) ABSTRACT

A device that reveals the biofilm present in the oral cavity of an individual. A device that reveals the biofilm and/or plaque present in the oral cavity of an individual in real time. A device that reveals plaque present in the oral cavity by illuminating plaque with a source of ultraviolet radiation.

### FIELD OF THE INVENTION

Disclosed herein is a device that reveals the biofilm present in the oral cavity of an individual. Also disclosed herein is a device that reveals the biofilm and/or plaque present in the oral cavity of an individual in real time. Also disclosed are methods of use of the biofilm reveal device.

### BACKGROUND OF THE INVENTION

Biofilm is a collection of one or more types of microorganisms that can grow on many different surfaces. One common example of a biofilm is dental plaque, which is a buildup of bacteria that forms on surfaces within the oral cavity.

Initially, dental plaque is a colorless deposit, but once it forms tartar, it is often brown or pale yellow. Tartar is a hard calcified deposit that forms by precipitation of minerals into the biofilm, which serves as a roughened surface that can host additional biofilm formation. This is known as calculus buildup. Calculus formation is associated with a number of clinical manifestations, including bad breath, receding gums and chronically inflamed gingiva. Brushing and flossing can remove plaque from which calculus forms; however, once formed, calculus is too firmly attached to be removed with a toothbrush.

As such, it is advantageous to quickly determine locations where plaque has begun to form since it can be easily removed by brushing and/or flossing. While a dental professional can easily identify biofilm and/or dental plaque, it can be challenging for an ordinary consumer to identify biofilm and/or dental plaque. Thus, it would be advantageous to for an individual to be able to identify the location of dental plaque in the oral cavity so that it can be more easily removed through brushing or flossing. The present invention is directed at devices that can allow an individual to assess dental plaque in real-time.

### SUMMARY OF THE INVENTION

Disclosed herein is a biofilm reveal device comprising (a) a container comprising a front side, (b) a source of ultraviolet radiation associated with the front side of the container and configured to provide ultraviolet radiation to an oral cavity of an individual, (c) a visor associated with the front side of the container configured to allow the individual to view inside the container; and (d) a reflection device within the container configured to allow the individual to view the oral cavity in contact with ultraviolet radiation.

Also disclosed herein is a method of revealing biofilm in an oral cavity of an individual comprising (a) providing the biofilm reveal device as described herein, (b) applying ultraviolet radiation to the oral cavity, and (c) viewing the oral cavity for illuminated portions.

### BRIEF DESCRIPTION OF THE DRAWINGS

FIG. 1 is a perspective view of a biofilm reveal device.

FIG. 2 is a side view of a biofilm reveal device.

FIG. 3 is a side view of a biofilm reveal device.

FIG. 4 is a top view of a biofilm reveal device.

FIG. 5 is a front view of a biofilm reveal device.

FIG. 6 is a perspective view of a visor of a biofilm reveal device.

FIG. 7 is a handle of a biofilm reveal device.

FIG. 8A is a reflective device.

FIG. 8B is a reflective device suitable for attachment within a biofilm reveal device.

### DETAILED DESCRIPTION OF THE INVENTION

The present invention is directed to a device that can reveal dental plaque to an individual in real-time without having to wait for an examination by a dental professional. While a dental professional can easily identify and remove dental plaque from surfaces in an oral cavity, a consumer might have difficulty identifying dental plaque in the oral cavity by merely looking in a mirror. Moreover, dental plaque can only be removed through brushing and/or flossing when it has recently attached in the oral cavity. The longer that dental plaque is in the oral cavity, the harder that it can be to remove as minerals precipitate into the biofilm to create tartar.

The present invention solves this problem by allowing a user to quickly assess biofilm in real-time using the disclosed device. The user can look into the device which comprises a reflective surface to reveal the user's oral cavity in contact with ultraviolet radiation. Plaque and/or biofilm, if present, will be illuminated by the electromagnetic and/or ultraviolet radiation. Additionally, a pigment can be applied to oral cavity, such as pigment that reflects visible light, such as light with wavelengths associated with the orange color. The pigment can improve the visibility of biofilm and/or plaque within the oral cavity.

Thus, the user can quickly assess whether biofilm/dental plaque are present, and if so, the exact location within the oral cavity to focus removal efforts prior to the development of tartar.

### Definitions

By “oral care composition”, as used herein, is meant a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice”, as used herein, includes tooth or subgingival-paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

As used herein, “translucent” is understood to describe a material that allows some electromagnetic radiation to pass through. A translucent material allows some light to pass through, such that some objects cannot be seen distinctly. Thus, a translucent material allows the consumer to see the material held within the translucent material, but in certain instances without much detail.

As used herein, “transparent” is understood to describe a material that allows nearly all electromagnetic radiation to pass through. A transparent material allows most of the light to pass through. Thus, a consumer will generally be able to see detailed images of other materials through transparent materials.

As used herein, “opaque” is understood to describe a material that does not allow nearly any light or electromagnetic radiation to pass through. Thus, a consumer will not generally be able to see materials through opaque materials.

As used herein, “electromagnetic radiation,” describes all of the energies released into space by stars or the sun. Electromagnetic radiation includes energy in the forms of waves and/or photons.

Electromagnetic radiation carries radiant energy. Electromagnetic radiation includes all electromagnetic radiation at every wavelength. Electromagnetic radiation includes, but is not limited to radio waves, microwaves, infrared radiation, visible light, ultraviolet radiation, x-ray, gamma rays, and/or mixtures thereof. Electromagnetic radiation can be provided by any source capable of producing electromagnetic radiation. Sources of electromagnetic radiation include, but are not limited to, a light emitting diode, incandescent bulb, a fluorescent bulb, a compact fluorescent bulb, a halogen bulb, a metal halide bulb, a high pressure sodium bulb, a low pressure sodium bulb, and/or a mercury vapor bulb. The electromagnetic radiation source can be a laser.

As used herein, “ultraviolet radiation,” or “ultraviolet light” describe electromagnetic radiation from any source with a wavelength from about 10 nm to about 500 nm.

As used herein, “visible radiation,” or “visible light” describe electromagnetic radiation from any source with a wavelength of from about 400 nm to about 750 nm.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The foregoing summary is not intended to define every aspect of the invention, and additional aspects are described in other sections. In addition, the invention includes, as an additional aspect, all embodiments of the invention narrower in scope in any way than the variations defined by specific paragraphs set forth herein. For example, certain aspects of the invention that are described as a genus, and it should be understood that every member of a genus is, individually, an aspect of the invention. Also, aspects described as a genus or selecting a member of a genus should be understood to embrace combinations of two or more members of the genus. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

Features of the compositions and methods are described below. Section headings are for convenience of reading and not intended to be limiting per se. The entire document is intended to be related as a unified disclosure, and it should be understood that all combinations of features described herein are contemplated, even if the combination of features are not found together in the same sentence, or paragraph, or section of this document. It will be understood that any feature of the methods or compounds described herein can be deleted, combined with, or substituted for, in whole or part, any other feature described herein.

### Plaque Reveal Device

The present invention is directed towards a plaque and/or biofilm reveal device (10) comprising a source of ultraviolet radiation, such as (30), and a means for allowing the user to visually assess the presence and/or amount of biofilm and/or plaque in the user's oral cavity, such as (20).

### Source of Electromagnetic Radiation

The biofilm reveal device (10) comprises a source of electromagnetic radiation. Electromagnetic radiation describes all of the energies released into space by stars or the sun. Electromagnetic radiation includes energy in the forms of waves and/or photons. Electromagnetic radiation carries radiant energy. Electromagnetic radiation includes all electromagnetic radiation at every wavelength. Electromagnetic radiation includes, but is not limited to radio waves, microwaves, infrared radiation, visible light, ultraviolet radiation, x-ray, gamma rays, and/or mixtures thereof.

Preferably, the biofilm reveal device comprises a source of ultraviolet radiation. While not wishing to being bound by theory, it is believed that ultraviolet radiation can illuminate biofilm in the oral cavity, thereby allowing the user to assess the amount of biofilm present in the oral cavity.

Electromagnetic radiation can be provided by any source capable of producing electromagnetic radiation. Sources of electromagnetic radiation include, but are not limited to, a light emitting diode, incandescent bulb, a fluorescent bulb, a compact fluorescent bulb, a halogen bulb, a metal halide bulb, a high pressure sodium bulb, a low pressure sodium bulb, and/or a mercury vapor bulb. The electromagnetic radiation source can be a laser.

The biofilm reveal device (10) can comprise a camera (30). The camera (30) can include a transparent and/or translucent window (31). The camera (30) can comprise a source of electromagnetic radiation, such as ultraviolent radiation. The camera (30) can include a visor (33), a raised edge, or a member configured for the placement of the bottom half of the face of the user. The visor (33) can have concave portions configured to accept the placement of the bottom portion of a user's face so that the electromagnetic radiation can be efficiently transmitted to the oral cavity of a user.

The camera (30) can be configured to illuminate the oral cavity of user with ultraviolent radiation and allow a user to view the oral cavity through the back side of the camera from a reflective device (50) within the biofilm reveal device (10).

The camera (30) can be associated with or removably attached to the biofilm reveal device (10). The camera (30) can have a gripping element (32) so that the camera can be removed or attached with the biofilm reveal device (10).

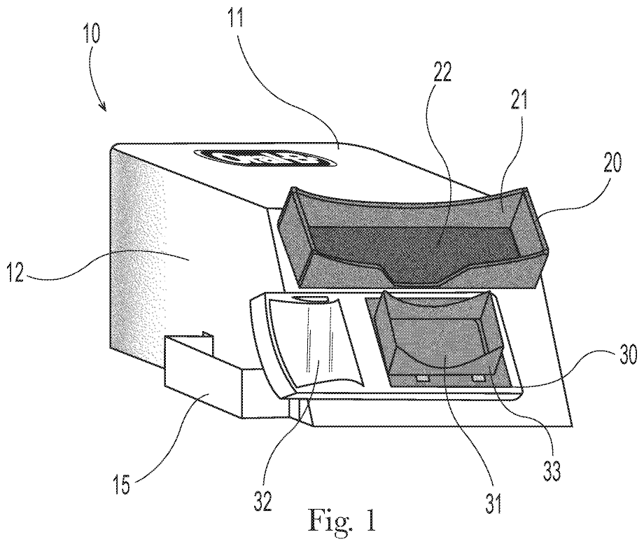

A suitable source of electromagnetic radiation, such as ultraviolet radiation, is shown in FIG. 1. The source of electromagnetic radiation can have a front side and a back side. The front side can provide the electromagnetic radiation, while the back side can be translucent or transparent to allow the area receiving ultraviolet radiation to be seen.

A suitable source of electromagnetic radiation can be found in KR20180104388, KR20170101589, and/or KR20180042005, which are herein incorporated by reference in their entirety. Each of these references discloses a camera that can provide a suitable source of electromagnetic radiation, such as ultraviolet radiation.

The wavelength of the ultraviolet radiation can be from about 10 nm to 500 nm, from about 200 nm to about 450 nm, from about 350 nm to about 450 nm, or about 405 nm.

### Container

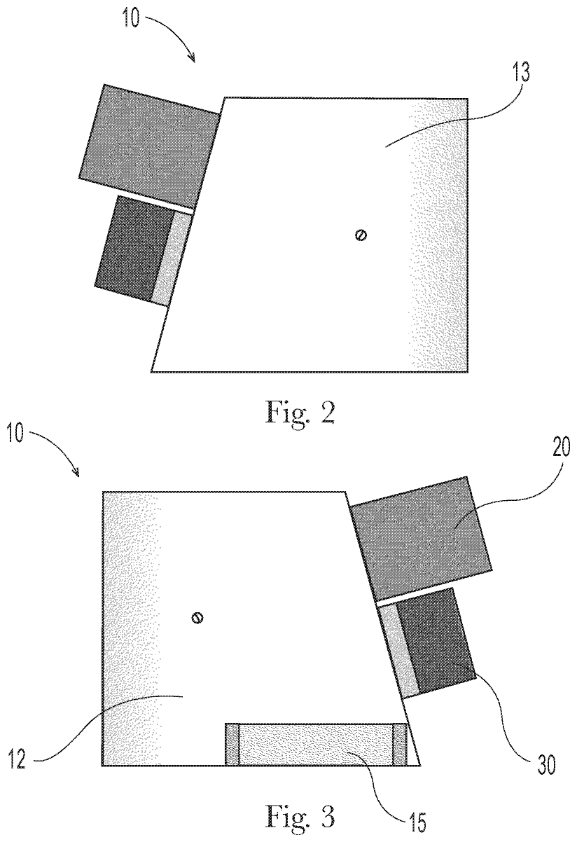

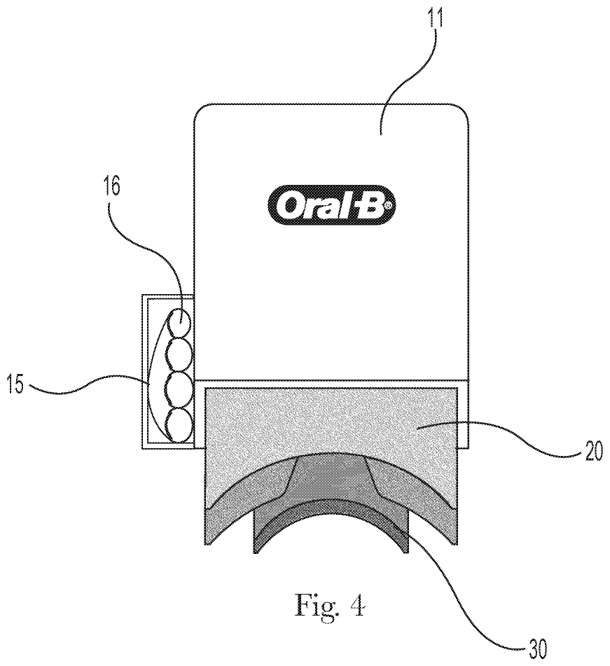

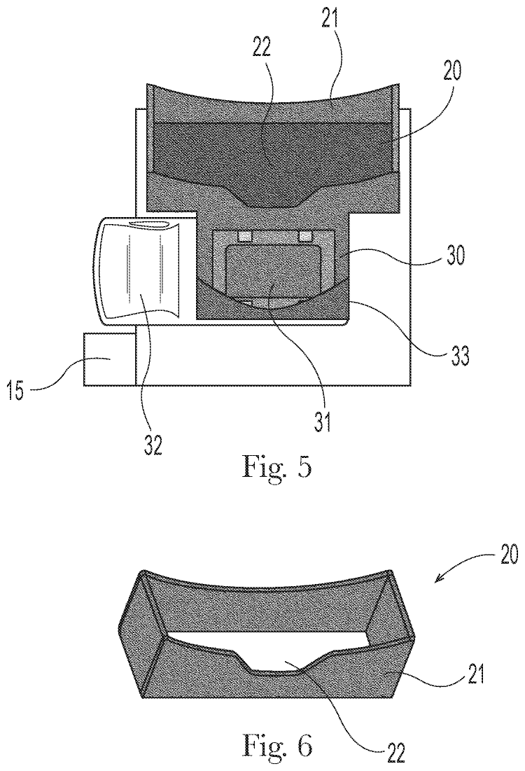

The biofilm reveal device can comprise an outer container, such as in FIG. 1. The outer container can comprise a front side (14), a left side (12), and a right side (13) (FIG. 2 3), a back side, a top side (11, FIG. 4), and a bottom side. The ultraviolet radiation source can be placed on the front side (14) of the outer container, such as in FIG. 1 and/or FIG. 5.

The outer container can have a width of from about 50 mm to about 500 mm, from about 100 mm to about 250 mm, or from about 150 mm to about 200 mm. The outer container can have a depth of from about 50 mm to about 500 mm, from about 100 nm to about 250 mm, or from about 150 mm to about 200 mm. The outer container can have a height of from about 50 mm to about 500mm, from about 100 mm to about 250 mm, or from about 150 mm to about 200 mm.

The outer container can be any suitable shape, such as for example, square, rectangular, trapezoidal, or any other polygon shape.

The outer container can be made from any suitable material, such as, for example, cardboard, acrylic, wood, metal, corrugated plastic, polyethylene terephthalate (PET), Glycol-modified Polyethylene Terephthalate (PETG), Oriented Polypropylene (OPP), Polyvinylchloride (PVC), Polyvinylidene Chloride (PVDC), Nylon, Polyethylene Terephthalate Polyester (PETP), and combinations thereof.

The outer container can comprise a viewing portion (20). The viewing portion (20) can comprise an opening (22) that can be configured to allow a user to view within the outer container. The opening (22) can allow the interior cavity of the outer container to be in fluid communication with the environment outside of the outer container. The opening (22) of the outer container can be square, rectangular, circular, oval, trapezoidal, or any other polygon shape. The opening (22) can have a length of from about 50 mm to about 200 mm, from about 75 mm to about 150 mm, or from about 100 mm to about 150 mm. The opening (22) can have a height of from about 25 mm to about 200 mm, from about 50 mm to about 100 mm, or from about 50 mm to about 75 mm.

The viewing portion (20) can also have a visor (21), as in FIG. 1-6, associated with the front of the container that can be configured for placement of the upper portion of face of the user, such as including the user's eyes, to minimize the escape of any electromagnetic radiation during assessment. The visor (21) associated with the opening can be have substantially the same length and height of the opening. The depth of the visor (21) can be from about 10 mm to about 100 mm, from about 25 mm to about 75 mm, or from about 50 mm to about 60 mm.

### Reflective Device

The biofilm reveal device (10) can comprise a reflective device (50) configured to allow the user to see the back side of the ultraviolet radiation source, such as in FIG. 8A and FIG. 8B.

The reflective device (50) can comprise a reflective surface (51), such as a mirror. The reflective surface (51) can be of any shape, such as circular, square, rectangular, triangular, etc., or size that fits within the outer container.

The reflective device (50) can have a means for adhering the reflective device (50) to the outer container. Suitable means include a hinge (52) and flap (53) that can be glued, taped, stapled, or the like to an inner surface of the outer container, such as in FIGS. 8A and 8B.

The reflective device (50) can include marketing information (54) to identify a particular brand or manufacturer, such as OralB® and/or Crest®.

The reflective device (50) can allow the user to look through the viewing portion (20) of the outer container and see the back side, if affixed with a screen and a camera, and/or through the ultraviolet radiation source, if affixed with a transparent and/or translucent surface, such as a lens, to visually assess the oral cavity contacted with the ultraviolet radiation source. This can reveal the presence of plaque and/or biofilm in the oral cavity to the user.

The outer container can have an interior cavity, which can be opaque. The interior cavity can be covered with a nonreflective material to improve the visibility of the oral cavity to the user. The nonreflective material can be a dark pigment. The nonreflective material can be a darkly colored tape, such as black tape.

### Handle

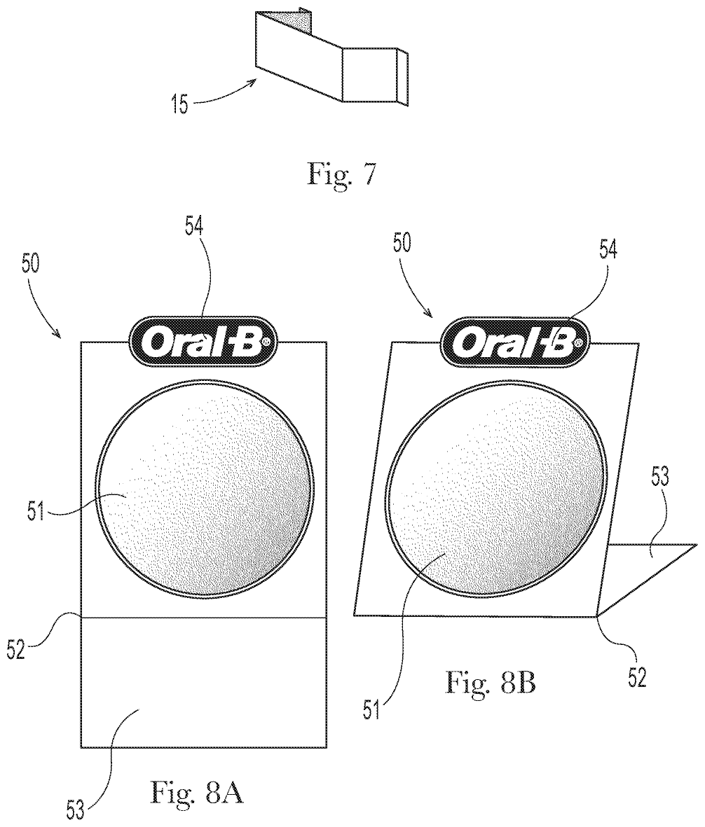

A handle (15), such as in FIG. 7 can be associated with the biofilm reveal device (10). The handle (15) can allow the user to hold the biofilm reveal device (10) up to their face so that biofilm in the oral cavity can be assessed. The handle (15) can include individual finger openings (16), such as in FIG. 4.

### Methods of Use

Also disclosed are methods of use of the biofilm and/or plaque reveal device. The biofilm and/or plaque reveal device, as described herein, can be provided. Next, ultraviolet radiation can be applied to the oral cavity. The user can place his or her face on the visor and align his or her oral cavity in front of the source of electromagnetic and/or ultraviolet radiation. The user can look through the opening and view the oral cavity contacted with electromagnetic and/or ultraviolet radiation by way of the reflective device. Plaque and/or biofilm, if present, will be illuminated by the electromagnetic and/or ultraviolet radiation. Additionally, a pigment can be applied to oral cavity, such as pigment that reflects visible light, such as light with wavelengths associated with the orange color. The pigment can improve the visibility of biofilm and/or plaque within the oral cavity.

FIG. 1 shows a biofilm reveal device (10). The biofilm reveal device (10) of FIG. 1 has an outer container with a top side (11), a left side (12), and a right side (13), a back side, and a bottom side. The biofilm reveal device (10) of FIG. 1 has a handle (15) configured for holding the biofilm reveal device (10) up to the face of a user. The biofilm reveal device (10) of FIG. 1 has a viewing portion (20) with an opening (22) and a visor (21) configured to accept the upper portion of the face of a user. The biofilm reveal device (10) of FIG. 1 has a camera (30) as the source of electromagnetic radiation, which in FIG. 1 is ultraviolet radiation. The camera (30) includes a window (31), such as a lens, so that the user can look through the back of the camera with the aid of an internal reflective device (50) to view the oral cavity in contact with ultraviolet radiation. The camera (30) of FIG. 1 has a gripping element (32) and a visor (33) configured to accept the placement of the bottom portion of a user's face so that the electromagnetic radiation can be efficiently transmitted to the oral cavity of a user.

FIGS. 2 and 3 are side views of biofilm reveal device (10) of FIG. 1. FIGS. 2 and 3 includes a viewing portion (20) and a camera (30). The biofilm reveal device (10) of FIGS. 2 and 3 includes a handle (15) configured for holding the biofilm reveal device (10) up to the face of a user.

FIG. 4 is a top view of a biofilm reveal device (10). The biofilm reveal device (10) of FIG. 4 includes a handle (15) with individual finger openings (16). Additionally, the biofilm reveal device (10) of FIG. 4 includes a viewing portion (20) and a camera (30).

FIG. 5 shows a biofilm reveal device (10). The biofilm reveal device (10) of FIG. 5 has a handle (15) configured for holding the biofilm reveal device (10) up to the face of a user. The biofilm reveal device (10) of FIG. 5 has a viewing portion (20) with an opening (22) and a visor (21) configured to accept the upper portion of the face of a user. The biofilm reveal device (10) of FIG. 5 has a camera (30) as the source of electromagnetic radiation, which in FIG. 5 is ultraviolet radiation. The camera (30) includes a window (31), such as a lens, so that the user can look through the back of the camera with the aid of an internal reflective device (50) to view the oral cavity in contact with ultraviolet radiation. The camera (30) of FIG. 5 has a gripping element (32) and a visor (33) configured to accept the placement of the bottom portion of a user's face so that the electromagnetic radiation can be efficiently transmitted to the oral cavity of a user.

FIG. 6 shows a viewing portion (20) with an opening (22) and a visor (21) configured to accept the upper portion of the face of a user.

FIG. 7 shows a handle (15) configured for holding the biofilm reveal device (10) up to the face of a user.

FIG. 8A shows a reflective device (50). The reflective device (50) of FIG. 8A can be placed in the outer container to allow the user to look through the camera (30) window (31) to view the oral cavity in contact with the ultraviolet radiation. The reflective device (50) of FIG. 8A includes a reflective surface (51), a hinge (52), and an unfolded flap (53). The reflective device (50) of FIG. 8B has a folded flap (53) to provide a surface to adhere within the outer container. The reflective device (50) of FIGS. 8A and 8B include marketing information (54).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A biofilm reveal device comprising:
(a) an outer container comprising a front side;
(b) a source of ultraviolet radiation associated with the front side of the outer container and configured to provide ultraviolet radiation to an oral cavity of an individual;
(c) a viewing portion;; and
(d) a reflection device within the container configured to allow the individual to view the oral cavity in contact with ultraviolet radiation.

2. The biofilm reveal device of claim 1, wherein the biofilm reveal device comprises a camera.

3. The biofilm reveal device of claim 2, wherein the camera comprises the source of ultraviolet radiation.

4. The biofilm reveal device of claim 2, wherein the camera comprises a gripping element.

5. The biofilm reveal device of claim 2, wherein the camera is removably attached to the front side of the outer container.

6. The biofilm reveal device of claim 1, wherein the reflection device comprises a hinge.

7. The biofilm reveal device of claim 1, wherein the reflection device is adhered to an interior surface of the outer container.

8. The biofilm reveal device of claim 1, wherein the outer container comprises a handle configured for holding the biofilm reveal device up to the user.

9. The biofilm reveal device of claim 1, wherein the outer container has a width of from about 50 mm to about 500 mm.

10. The biofilm reveal device of claim 1, wherein the outer container has a height of from about 50 mm to about 500 mm.

11. The biofilm reveal device of claim 1, wherein the viewing portion comprises a first visor associated with the front side of the outer container configured to allow the individual to view inside the outer container.

12. The biofilm reveal device of claim 11, wherein the viewing portion comprises an opening.

13. The biofilm reveal device of claim 12, wherein the opening is square, rectangular, circular, oval, trapezoidal, or any other polygon shape.

14. The biofilm reveal device of claim 12, wherein the opening has a length of from about 50 mm to about 200 mm.

15. The biofilm reveal device of claim 14, wherein the opening has a height of from about 50 mm to about 100 mm.

16. The biofilm reveal device of claim 11, wherein the first visor has a depth of from about 25 mm to about 75 mm.

17. The biofilm reveal device of claim 8, wherein the handle comprises individual finger openings.

18. The biofilm reveal device of claim 1, wherein the camera comprises a second visor.

19. The biofilm reveal device of claim 18, wherein the camera is configured to illuminate the oral cavity of user with ultraviolent radiation and allow a user to view the oral cavity through the back side of the camera from a reflective device within the biofilm reveal device.

20. A method of revealing biofilm in an oral cavity of an individual comprising:
(a) providing the biofilm reveal device of claim 1;
(b) applying ultraviolet radiation to the oral cavity; and
(c) viewing the oral cavity for illuminated portions.
